(19) United States
(12) Patent Application Publication
(10) Pub. No.: US 2010/0324564 A1
(43) Pub. Date: Dec. 23, 2010

(54) PARTICLES, GRAINS OR GRANULES FOR THE TREATMENT OF AN INJURED VERTEBRA

(76) Inventors: Lars Magnus Bjursten, Limhamn (SE); Bengt Mjoberg, Ystad (SE)

Correspondence Address: HARNESS, DICKEY & PIERCE, P.L.C., P.O. BOX 8910, RESTON, VA, 20195, US

(21) Appl. No.: 12/733,882

(22) PCT Filed: Nov. 5, 2008
(86) PCT No.: PCT/SE2008/000629

§ 371 Date: Aug. 30, 2010

Related U.S. Application Data
(60) Provisional application No. 60/996,216, filed on Nov. 6, 2007.

(30) Foreign Application Priority Data
Nov. 6, 2007 (SE) 0702454.0

(51) Int. Cl. A61B 17/58; A61P 19/10; A61K 9/14; A61K 33/24; A61K 38/18; A61K 31/663; A61K 31/194; A61K 31/366; A61K 31/7036; A61K 38/39; B32B 5/16

(52) U.S. Cl. 606/92; 424/489; 424/617; 514/8.8; 514/75; 514/574; 514/460; 514/40; 514/16.9; 428/402

(57) ABSTRACT

A method is provided for treatment of a condition including at least one weakened vertebra or one compression fracture of a vertebra, wherein in at least one embodiment, the vertebra is injected by vertebroplasty or kyphoplasty with particles, grains or granules or a mixture thereof, or an implant suspension including at least one resorbable fluid vehicle and particles, grains or granules or a mixture thereof, wherein the particles, grains or granules individually have a mean length from one side to the opposite side, through a geometrical centre, of up to 5 mm. Moreover, at least one embodiment of the present invention also provides use of the particular or granular material disclosed above, for the manufacture of a medicament for the vertebroplastic treatment or kyphoplastic treatment of a condition including at least one weakened vertebra or one compression fracture of a vertebra. Finally, at least one embodiment of the present invention provides the particular or granular material disclosed above, for the vertebroplastic treatment or kyphoplastic treatment of a condition including at least one weakened vertebra or one compression fracture of a vertebra, such as a condition like osteoporosis.

### FIELD OF THE INVENTION

The present invention relates to a method for treatment of a condition comprising at least one weakened vertebra or one compression fracture of a vertebra. Moreover, the present invention relates to use of particles, grains or granules or a mixture thereof, or an implant suspension comprising particles, grains or granules or a mixture thereof and a resorbable fluid vehicle, for the manufacture of a medicament for the vertebroplastic treatment or kyphoplastic treatment of a condition comprising at least one weakened vertebra or one compression fracture of a vertebra. The present invention is also directed to particles, grains and granules or a mixture thereof, or an implant suspension comprising such, for the vertebroplastic treatment or kyphoplastic treatment of a condition according to above.

### BACKGROUND OF THE INVENTION

A spinal fracture occurs when one of the bones in the spinal column breaks. This type of fracture is also known as a vertebral compression fracture due to the fact that the bone that breaks (the vertebral body) often cracks and collapses, i.e. becomes compressed. Certain diseases, such as osteoporosis or cancer, are known to cause loss of bone mass and changes in bone structure, which makes these bones brittle and weak. Genetic factors and certain lifestyles, such as a low calcium diet, can also damage the bones. Over time, the vertebral bodies can become so weak that normal activities, such as bending over, can cause a spinal fracture.

The adult spine is a column of bones that protects the spinal cord and enables humans to stand upright. Each bone segment of the spine is referred to as a vertebra. At the front of each vertebra is a block of bone called the vertebral body. The vertebral body consists of an inner core of soft cancellous bone, surrounded by a thin outer layer of hard cortical bone. Vertebrae in the cervical, thoracic and lumbar regions are separated from each other and cushioned by a rubbery soft tissue called the intervertebral disc. Segments of bone that extend outward at the back of each cervical, thoracic and lumbar vertebral body surround and protect the spinal cord and its nerve roots. The vertebral bodies in the thoracic and lumbar regions have the greatest risk for fracture due to osteoporosis. Cancer and benign tumors can lead to fracture of the cervical, thoracic and lumbar vertebrae.

If bones are healthy, it takes an injury of considerable force to cause a fracture. A spinal fracture occurs when one of the bones of the spinal column fractures or collapses. When more than one spinal fracture occurs, loss of height or spinal deformities may result. Certain conditions, including osteoporosis, cancer and long-term use of steroids or other drugs, can make bones fragile and more likely to break. In older adults, osteoporosis is, however, the primary cause of spinal fractures.

Osteoporosis is a disease where loss of bone mass and loss of bone quality makes someone more likely to fracture. In osteoporosis, the balance between the normal breakdown and rebuilding of bone is disrupted. Bone breakdown exceeds bone repair. Over time, this imbalance causes the areas of weak bone to accumulate to the point that the bone breaks after minor trauma, such as stepping off a curb or rolling over in bed. Aging tends bones to weaken. This weakening is related to a number of factors, including a decline in our ability to produce vitamin D and absorb calcium, as well as a decrease in our levels of sex hormones. Osteoporosis related to aging and to menopause is called primary osteoporosis. Certain drugs and many diseases affect bone health, leading to secondary osteoporosis. Both types of osteoporosis increase the risk for fracture.

There exists different ways of surgically treating a vertebral compression fracture today. The common way is the usage of bone cement. Vertebroplasty involves the infiltration of viscous bone cement into the injured or fractured vertebra during fluoroscopy. The other known common surgery type is kyphoplasty, which also involves the infiltration of viscous bone cement, but in this case another additional step is performed before. A surgical empty balloon is in this case inserted into the vertebra. Once in place the balloon is filled during pressure and volume control with a radiopaque liquid so that the vertebra is possible to control in the sense of regaining its normal shape. Thereafter the balloon is surgically removed and the formed cavity is filled with bone cement, just as in the case of vertebroplasty.

There are problems with these existing techniques. One of the most important and serious problems is the risk of leakage of bone cement from a treated vertebra and into e.g. segmental veins and to the lungs and into the spinal canal. It has been suggested that kyphoplasty should involve a smaller risk of these leakages than the standard vertebroplasty, due to the fact that it is possible to use a lower pressure with kyphoplasty when injecting bone cement into the formed cavity, but this has not yet been proven. In fact, studies, at this time show that both of the treatments yield a leakage of cement outside the vertebra in 10 percent or more of the cases of treatment.

Grain or granule implants are known from state of the art. One example is e.g. granules of hydroxyapatite. Other examples are metal or metal alloy implants. For references of implants where the metal titanium could be used, U.S. Pat. No. 5,015,256 (Bruce et al) discloses means and a method for fixing an elongate prosthesis, such as the stem of a femoral prosthesis, to living tissue which defines a cavity in which a length of the prosthesis is received with a gap to the boundary of the cavity. Essentially the entire gap is filled with loose, but packed grains of a biocompatible material, said grains interlocking. As an example of granular material, titanium is mentioned, and the grains are stated to be irregular, essentially non-elastic and preferably porous, the latter property being said to promote growth of bone tissue which has grown from the osseous wall. The grain interlocking has been achieved by vibrating the stem into a bed of grains housed in said cavity and by a final blow on the stem.

Another example of grain implants is disclosed in WO00/64504 (Bruce et al), which discloses a biocompatible, plastic or essentially non-elastic, porous body, such as a grain, with continuous porosity, the openings of cavities and the passages interconnecting them having a width of >about 50 μm for bone tissue. The term “continuous” is said to mean a porosity which allows bone tissue to grow through the porous body. The porous body may be of titanium. Moreover, the Swedish patent application no 0700457-5 discloses an implant with anti-inflammatory or antibacterial effects, or both, the implant being intended for implantation in a human or an animal body, the implant comprising at least one porous granule or grain, wherein the at least one porous granule or grain
comprises titanium, one or more titanium oxides or titanium alloy and has a titanium oxide layer on its surface;
has a mean length from one side to the opposite side, through a geometrical centre, of up to 5 mm; and
has a mean specific surface area of at least 0.15 m²/g according to the BET method.

One object of the present invention is to improve the existing technologies for treatment of a weakened vertebra or a compression fracture of a vertebra, e.g. due to osteoporosis. Such existing technologies are vertebroplasty and kyphoplasty by use of bone cement. Another important object is to minimise the risk of leakage of any dangerous material from the vertebra and e.g. into the spinal canal after such a treatment. One object of the present invention is also to enhance the stability of the treated vertebra.

### SUMMARY OF THE INVENTION

The objects above are solved by the present invention which provides a method for treatment of a condition comprising at least one weakened vertebra or one compression fracture of a vertebra, wherein the vertebra is injected by vertebroplasty with particles, grains or granules or a mixture thereof, or an implant suspension comprising at least one resorbable fluid vehicle and particles, grains or granules or a mixture thereof, wherein the particles, grains or granules individually have a mean length from one side to the opposite side, through a geometrical centre, of up to 5 mm.

### DETAILED DESCRIPTION OF THE INVENTION

Due to the fact that the method for treatment of a condition comprising a weakened vertebra or a compression fracture of a vertebra according to the present invention involves the insertion of particles, grains or granules, there does not exist any risk of leakage of any dangerous material, such as bone cement, out into e.g. the spinal canal.

According to the present invention either particles, grains or granules or a mixture thereof, or an implant suspension comprising at least one resorbable fluid vehicle and particles, grains or granules or a mixture thereof, are inserted into at least one vertebra. This should be interpreted as the fact that it is possible according to the present invention to insert particles, grains or granules or a mixture thereof in themselves, i.e. more or less dry, or in an implant suspension additionally comprising at least one fluid vehicle. The scope of the present invention should be interpreted as also covering particles, grains or granules or a mixture thereof being only moistened, e.g. on their surfaces, by a fluid, such as any fluid disclosed as one of the possible fluid vehicles according to the present invention.

As mentioned above there may be some beneficial effects of using a surgical balloon, such as in a kyphoplastic treatment, for the treatment of a condition comprising a weakened vertebra or a compression fracture of a vertebra. Therefore, according to one specific embodiment of the present invention there is provided a method for treatment of a condition comprising at least one weakened vertebra or one compression fracture of a vertebra, wherein kyphoplasty with a surgical balloon is performed on the vertebra and a cavity formed inside of the vertebra, after the balloon has been surgically removed, is injected with particles, grains or granules or a mixture thereof, or an implant suspension comprising at least one resorbable fluid vehicle and particles, grains or granules or a mixture thereof, wherein the particles, grains or granules individually have a mean length from one side to the opposite side, through a geometrical centre, of up to 5 mm.

It is important to realise that the expressions “vertebroplasty” and “kyphoplasty”, respectively, in relation to the present description and invention should be interpreted as to include all of the steps performed with a standard vertebroplastic treatment (vertebroplasty) and kyphoplastic treatment (kyphoplasty), respectively, except for the step of inserting bone cement into the vertebra.

The particles, grains or granules according to the present invention may be comprised of different type of materials. Therefore, according to one specific embodiment of the present invention, the particles, grains or granules are of any type of material that is a polymer, ceram, metal-ceramic mixture (composite), metal, metal alloy, metal oxide or a mixture thereof. Specific examples are e.g. hydroxyl apatite (hydroxyapatite), other calcium phosphates such as tricalcium phosphate, or metals like tantalum. According to another specific embodiment of the present invention, the particles, grains or granules comprise at least one bone growth promoting metal, metal oxide or metal alloy or a mixture thereof, such as titanium, one or more titanium oxides or titanium alloy or a mixture thereof. Tantalum is another metal which also could be categorised as bone growth promoting in some sense.

The shape of the particles, grains or granules according to the present invention may in some cases be of specific interest in relation to the application and use thereof. Spherical particles, grains and granules are normally easy to inject from e.g. an introducer with a troacar or any other type of injector. The sizes of the particles, grains or granules can be optimised in relation to different applications, but in general firstly of course the sizes are below the diameter of e.g. the diameter of the used introducer, and secondly individually of sizes in relation to each other that promote bone growth in between different particles or grains. In general bone cells need at least 50 μm in space size to be able to grow through such a space, and hence cannot grow through e.g. pores of less diameter. Therefore, in those applications where bone growth and ingrowth in between the particles, grains or granules are important, there should exist cavities in between these particles, grains or granules that are at least 50 μm in size, normally between 100-200 μm in size.

Moreover, in the applications mentioned above, the particles, grains or granules may naturally in some cases beneficially have bone inducting surfaces, e.g. in the case of injection directly into the vertebra. Therefore, according to one embodiment of the present invention the particles, grains or granules are spherical and optionally individually have a bone inducting surface. The surfaces may in some cases comprise pores which creates an environment which is bone inducting or bone growth promoting. Increasing the amount of pores is also a way to enhance the specific surface area, which sometimes is of interest in relation to increased bone growth and ingrowth effect. Another way of increasing the specific surface area is to provide nanotubes of microstructure on the surfaces of the particles, grains or granules. Therefore, according to one embodiment of the present invention, the particles, grains or granules are optionally spherical and are additionally provided with nanotubes of titanium oxide on their surfaces. Nanotubes of titanium oxide can be produced and provided on particles, grains and granules according to the present invention by anodic oxidation of these. This is further explained below.

According to another embodiment of the present invention, there is provided a method for treatment according to the present invention, wherein a final additional sealing injection step is performed last, in which sealing injection step at least one mesh and/or irregular particles, grains or granules, optionally of titanium, one or more titanium oxides or titanium alloy or a mixture thereof, are injected into the vertebra or the cavity formed inside of the vertebra to form a sealing. A mesh may in some cases be very useful to lock the particles, grains or granules already injected, and hence create a sealing. Irregular particles, grains or granules are possible to use accordingly. A combination of irregular particles, grains or granules and a mesh is also possible. All in all, a rigid overall structure can be achieved by using a mesh and/or irregular particles, grains or granules or a combination thereof.

Irregularity and porosity of particles, grains and granules are important parameters in relation to bone ingrowth and growth of bone cells. Therefore, according to one specific embodiment of the present invention the particles, grains or granules are irregular and optionally porous. In relation to the present invention these parameters are disclosed further below.

The implant suspension according to the present invention comprises at least one resorbable fluid vehicle, that is at least one resorbable liquid component. This at least one fluid vehicle is designed to disappear once inside of the body, such as the human or animal body. Hence, this fluid vehicle is totally different in comparison to e.g. a bone cement which is not resorbable in the host body. According to one specific embodiment of the present invention, the at least one resorbable fluid vehicle is chosen from the group consisting of NaCl (aq), hyaluronic acid, PEG, propylene glycole alginate (PGA), titanium peroxy gel, methyl cellulose, carbomethyl cellulose, dextran, a high viscous polymeric gel and a protein solution, or a combination thereof. According to another embodiment of the present invention, the implant suspension is a gel having a melting temperature above ambient temperature and below 37° C. (body temperature), which gel optionally comprises at least one of NaCl (aq), hyaluronic acid, PEG, propylene glycole alginate (PGA), titanium peroxy gel, methyl cellulose, carbomethyl cellulose, dextran, a high viscous polymeric gel, or a protein solution. This gel may be particularly useful in view of the fact that the gel and its containing particles, grains or granules will be easy to inject into a human or animal body when it is in a gel solid state at ambient temperature, but at the same time the gel becomes liquid at a normal body temperature making it readily and rapidly resorbable. Examples of such gels having that range of melting temperature may e.g. comprise hyaluronic acid in the right concentration for that gel to dissolve when being injected into a human or animal body.

Titanium is a metal having beneficial properties in contact with the biological host tissue, such as e.g. anti-inflammatory and/or antibacterial effects. As mentioned in the Swedish patent application no 0700457-5, there are a couple of important factors for achieving a grain or granule with enhanced anti-inflammatory and/or antibacterial effects. One of these factors or parameters is a high specific surface area of the grains or granules. Firstly, the size or diameter of the grain or granule affects the specific surface area, where the specific surface area for a perfect sphere decreases proportionally with the increase of the diameter, i.e. a 10 times increase of the original diameter decreases the specific surface area to 1/10 of the original specific surface area. This implies that the same volume or weight of smaller grains or granules have a higher surface area than larger ones. However, porosity is also of great importance for the specific surface area. With two identical grains in size, the one of them having many small pores has a larger specific surface area but smaller total pore volume in comparison to the other one with larger pores.

Thirdly, the irregularity is important. An irregular body has a higher specific surface area than a smooth body. As such, and in the two most opposite cases, an irregular flake of the same weight as a perfect smooth sphere has a much higher specific surface area than the sphere. In relation to the present invention, it could of course in some cases be preferable to provide particles, grains or granules having enhanced anti-inflammatory and/or antibacterial effects due to the fact that the grains or granules will be inserted into a human or animal body. Therefore, the particles, grains or granules according to the present invention are in some cases preferably irregular, both in relation to the surface of the particles, grains or granules as well as the surface of the pores of these grains or granules. Moreover, as mentioned, porous grains or granules could in some specific cases be preferable with the present invention due to the fact that porosity is a direct indication of the magnitude of the specific surface area.

As mentioned in the Swedish patent application no 0700457-5, the inventors, in an effort to take advantage of the beneficial properties of titanium as much as possible as well as produce a grain or granule with extremely high surface area, investigated crude spongy titanium from different suppliers, regarding e.g. their porosity. These investigations showed that crude titanium sponge produced by the well-known Hunter process or Kroll process is potentially a good candidate as crude material for forming an implant body (grain or granule) with such aimed properties, that is property to allow growth and ingrowth of tissue (bone; bone regeneration) as well as a property to have a bactericidal and anti-inflammatory effect, when placed in living tissue.

Other existing techniques suitable to use when producing the crude titanium/titanium alloy material, that is as sponges or blocks, are e.g. a direct foaming technique as gel casting, or other wet processing methods as a replication technique and a rapid prototyping technique.

The value of the specific surface area of grains or granules of titanium oxide/oxides, titanium or titanium alloy according to the Swedish patent application no 0700457-5 has proven to be an direct indication on the anti-inflammatory or antibacterial effects of the grains or granules.

The inventors found out that bodies of titanium sponge have a far better antibacterial and anti-inflammatory effect than bodies of non-porous titanium, which made them understand that effective surface area of a titanium body is a very important determining factor for good anti-bacterial and anti-inflammatory effect of titanium implants.

Both a sponge and small particles according to the invention of the Swedish patent application no 0700457-5 may possess antibacterial and anti-inflammatory effects. This proves that porosity is not the only determining factor for these effects, but size as well as irregularity are important as well. As mentioned, all of these three parameters determine the specific surface area, which is a measurement in direct correlation with the antibacterial and anti-inflammatory effects of an implant according to the Swedish patent application no 0700457-5.

However, it has turned out that increasing the surface area of a titanium implant by disintegrating (crushing) it to smaller pieces does not consistently bring about an improved antibacterial/anti-inflammatory effect.

Still further, the inventors of the invention disclosed in the Swedish patent application no 0700457-5 have surprisingly found that not only the size and the specific surface area of the titanium or titanium alloy body are determining for antibacterial and anti-inflammatory effects, but also other factors and conditions presented below are of great interest. For example could these effects be enhanced by binding or attaching other substances with specific properties to the implant according to the invention of the Swedish patent application no 0700457-5.

To summarise, the implant according to the Swedish patent application no 0700457-5, as well as the particles, grains or granules according to specific embodiments of the present invention, i.e. in the case of titanium, titanium oxide or titanium alloy, possess enhanced antibacterial and anti-inflammatory effects which are related to the specific features in terms of size, irregularity and porosity. All of these features determine the value of the specific surface area of the implant.

In this application the expression “implant” has the form of several particles, grains or granules parted from each other or is an agglomerate of particles, grains and/or granules, bonded together or not. Different expressions for the implant are used through out the description. Examples meaning the same thing in this sense are the expressions “granules” and “grains”.

The inventors of the invention disclosed in the Swedish patent application no 0700457-5 have found that for achieving effective antibacterial and anti-inflammatory effects, the implant must have a mean length from one side to the opposite side, through a geometrical centre, (referred to as the diameter in some cases) of maximum up to 5 mm, preferably from 200 μm and up to 2 mm. This is also applicable for an agglomerate where the particles, grains or granules are bonded together.

The particles and grains of the invention may, as mentioned, have an irregular shape, and with diameter is meant the longest axis length of two opposite points on a cross section of the grain. It is important to understand that the particles, grains or granules do not have to have the shape of a sphere. In fact, the irregularity of the implants is in some cases an important feature, and shapes that are more irregular, such as e.g. flakes, spikes, chips or similar or combinations thereof are possible and sometimes preferred.

According to one embodiment of the present invention, in relation to what is disclosed above of the size of particles, grains or granules, said particles:
have a mean length from one side to the opposite side, through a geometrical centre, of <200 μm; and
   said grains or granules:
have a mean length from one side to the opposite side, through a geometrical centre, of at least 200 μm and up to 2 mm.

This different “diameter” sizes define the normal difference between the meaning of the expressions “particles” and “grains” or “granules”.

According to another specific embodiment of the present invention the grains or granules have a mean specific surface area of at least 0.15 m²/g according to the BET method.

Below, the BET method for measuring the specific surface area will be described.

### Surface Area by Gas Adsorption

Surface area by gas adsorption is a measure of the exposed surface of a material reported in terms of square meters per gram. This most common model for assessing surface area is referred to as BET (Brunauer, Emmet and Teller) surface area or simply BET number. The key for obtaining a reliable, repeatable surface area result is to prepare the sample properly. Samples are prepared or degassed by applying some combination of heat, vacuum and/or flowing gas. This removes previously adsorbed contaminants from the surface and from the pores. Failure to remove these components effectively may result in erroneous data. Then the sample is cooled to cryogenic temperature, and an adsorptive gas (typically N₂) is admitted to the sample tube in controlled increments. After each dose, the pressure is allowed to equilibrate, and the quantity of gas adsorbed is calculated. The gas volume adsorbed at each pressure defines an adsorption isotherm. The quantity of gas required to form a monolayer over the surface of the solid is determined from the isotherm. The external surface area or BET surface area can be determined from the area covered by each adsorbed gas molecule known and the monolayer capacity known.

Firstly, isolated sites on the sample surface begin to adsorb gas molecules. Secondly, as gas pressure increases, coverage of gas molecules increases to form a monolayer. BET equation is used to calculate the surface area in this secondary stage. Thirdly, increasing gas pressure causes multilayer coverage. Smaller pores fill first. Still higher pressure then causes cornplete coverage of the sample and fill all of the pores. Pore diameter, volume and distribution calculation can be made by using the BJH (Barrett, Joyner, Halenda) method for surface calculation.

Multiple point equipment is used for determination of the pore size distribution in the secondary stage as well as the final stage.

### The Gemini Principle and Instrumentation

The Gemini uses an adaptive rate, static volumetric technique of operation. It is the first gas sorption method, which adapts the required rate at which gas is supplied for equilibration. The Gemini has two gas reservoirs, which are filled with equal volumes of the desired adsorptive, usually nitrogen. From the reservoirs, gas is dosed into the sample and balance tubes. A trans-ducer on the sample side monitors for the target pressure. As the sample adsorbs gas, the pressure would tend to decrease in the sample tube, but a first transducer causes a fast response servo valve to hold the pressure constant. A second transducer located between the sample and balance tubes detects any pressure difference between the two tubes and causes another servo valve to balance the pressures in both tubes. A third pressure transducer monitors the pressure between the two reservoirs to determine the amount of gas that is adsorbed on the sample. This method of dosing and accounting for the volume of gas uptake enables the Gemini to produce highly accurate, highly reproducible results in the minimum time.

According to the present invention, the specific surface area of the different particles, grains or granules is possible to measure according to the BET method, e.g. by using multiple point determination (Micromeritics Gemini 2360).

The determination of specific surface area according to this method is in the summary above, as well as hereinafter, described as determination according to the BET method.

There exist possible ways to enhance the desired structure of the granule in relation to irregularity, enhanced porosity and specific surface area. This is possible to perform by etching or roughening, where these two methods sometimes are used in combination. One feature distinguishing the particles or grains according to certain embodiments is as mentioned the irregular shape, and hence, roughening can be an aid to form or enhance this irregular shape of the particles or grains.

There exist different types of chemicals which can be used for etching. Some of them have additional beneficial effects to enhance the anti-inflammatory and/or antibacterial effects. One example is peroxides, e.g. a hydrogen peroxide solution.

The reaction product of hydrogen peroxide and metallic titanium, that is a titanium peroxy radical gel, is disclosed as an anti-inflammatory oxidizing agent in the international patent publication WO89/06548 (Bjursten et al.). WO89/06548 discloses the reaction gel product as a coating on implants made of titanium or with a titanium coating, but these implants are not in any way similar to the grains according to the present invention. The implants according to WO89/06548, however, are not grains or granules as the case in the present invention, but solid implants for a specific function, as e.g. a titanium screw. In other words, an implant according to WO89/06548 could be categorised as a “whole-body prosthesis”. When stating the implant according to the invention as particles, grains or granules, it is important to understand the differences between the implants according to the present invention and “whole-body implants” according to state of the art. These “whole-body implants” are e.g. a titanium screw or a tooth, and not at all particles, grains or granules, and common for all of these “whole-body implants” are that they have at least one fastening or fixing element, which is not the case with particles, grains or granules according to the present invention.

The surface, including the pore surfaces, of the particles and/or grains according to the invention may be exposed to peroxides, as mentioned, before the particles and/or grains are implanted, whereby the antibacterial effect of the implant is enhanced.

There exist other chemicals which could be used for similar purposes, that is etching and enhancing the anti-inflammatory and/or antibacterial effects. Therefore, according to one embodiment of the present invention there is provided a method for treatment, wherein the particles, grains or granules are pre-treated with at least one fluoric compound, hydrochloric acid, sulphuric acid, phosphorous acid, a peroxide compound chosen from the group consisting of hydrogen peroxide (H₂O₂) and organic peroxides, or oxalic acid, or a combination thereof, or dry etched with fluorinated or chlorinated gases. This pre-treatment is normally made to enhance the specific surface area of the particles, grains or granules, which in some cases are porous, and/or to oxidise these particles, grains or granules.

According to one specific embodiment of the invention, the fluoric compound used is any type of fluoric acid, hydrofluoric acid in combination with nitric acid, ammonium fluoride, ammonium bifluoride (also in combination with nitric acid), or hydrogen fluoride (HF).

The treatment conditions for a possible titanium/titanium oxide/titanium alloy particle, grain or granule to use vary in relation to concentration, time and temperature, depending on the specific chemical used as well, and there are many different combinations possible to use.

According to one specific embodiment, the concentration of the chemical used in the treatment above is from 0.05 to 1.0% for fluoride acids, from 0.5 to 30.0% for hydrogen peroxides and from 0.2 to 20.0% for oxalic acids. According to one specific example, the concentration is about 0.2% for fluoride acids, about 30% for hydrogen peroxides and about 10% for oxalic acids.

Oxidation is also an effective method for changing the chemistry of the particles or grains. Oxidation could be used for different purposes, e.g. for increasing the specific surface area of the particles or grains, for enhancing the amount of titanium oxide and thereby the possible antibacterial and/or anti-inflammatory effect or for altering the appearance of the particles or grains, or a combination thereof.

According to one embodiment of the present invention, the particles, grains or granules are pre-treated by oxidation by heat treatment in oxidising atmosphere at temperatures between 20 and 1000° C. and/or by electrochemical procedure.

According to another specific embodiment, the particles, grains or granules are pre-treated by anodical oxidation using spark erosion to increase the surface area.

According to yet another specific embodiment of the present invention, the particles, grains or granules have been produced according to a spark erosion procedure where the particles, grains or granules have been brought in contact with an anode using a flexible net or porous sponge-like structure.

Anodic spark deposition of titanium or titanium alloy implants is e.g. described in “Osteointegration of titanium and its alloys by anodic spark deposition and other electrochemical techniques: A review” in Journal of Applied Biomaterials & Biomechanics 2003; 1: 91-107 by R. Chiesa et al. The deposition according to the document above was achieved by potentiostatic polarization of the cathode with a potential in the range of −1 500 to −1 300 mV (vs. SCE). The implants described in this document are homogenous implants with surfaces modified by spark deposition and thus fundamentally different from the particles, grains or granules according to the present invention.

According to another specific embodiment of the present invention, there is provided a method for treatment according the present invention, wherein the particles, grains or granules are provided with nanotubes of titanium oxide, by a pre-treatment involving anodic oxidation, on their surfaces. This pre-treatment comprising anodic oxidation can e.g. be performed according to Cai, Paulose, et al. in “The effect of electrolyte composition on the fabrication of self-organized titanium oxide nanotube arrays by anodic oxidation”, J Mater Res 2005 20; 1:230-6, or according to Macak, Tsuchiya et al. in “Smooth Anodic TiO₂ Nanotubes”, Angewandte Chemie: a journal of the Gesellschaft Deutscher Chemiker, Int. ed. 44; 2005; 7463-65, which both in full are hereby incorporated by reference.

The provision of nanotubes of titanium oxide on the surfaces of particles, grains or granules according to the present invention may be of importance to achieve a high specific surface area and hence surfaces which are highly bone inducting. As mentioned in the references above it is possible to produce nanotubes with a length of up to 7 μm using the specific conditions disclosed in those references. Therefore, it should, by use of some modifications, be possible to produce nanotubes with an individual length of up to about 10 μm. In any case, nanotubes of a length of 1-5 μm are without specific optimisation possible to achieve, and hence possible to provide on the surfaces of the particles, grains or granules according to the present invention by anodic oxidation. Nanotubes of a length of up to about 1 μm have good mechanical properties, so this length is quite sufficient in relation to this embodiment of the present invention.

Anodic oxidation is performed in some kind of electrolyte. Hydrofluoric acid (HF) is a component which is often comprised in the electrolyte. However, if the length of the produced nanotubes is of great importance, e.g. electrolyte solutions containing potassium fluoride, sodium fluoride, glycerol, (NH₄)₂SO₄ or (NH₄)F may be suitably used for production of longer nanotubes of titanium oxide. Therefore, according to one specific embodiment of the present invention the particles, grains or granules are provided with nanotubes of titanium oxide, by a pre-treatment involving anodic oxidation, on their surfaces, in an electrolyte solution comprising hydrofluoric acid (HF), potassium fluoride (KF), sodium fluoride (NaF), glycerol, (NH₄)₂SO₄ or (NH₄)F or a combination thereof.

The electrolyte solutions may of course contain other chemicals as well and these may be added for different reasons. For example, pH adjustment of the electrolyte solutions may be performed by the addition of e.g. sulphuric acid, sodium hydroxide, sodium hydrogen sulphate, citric acid, among others.

It has previously been found that the crystalline isoforms, anatase and rutile, of titanium oxide are more efficient than the amorphous titanium oxide in catalytic reactions, as the source of the anti-inflammatory and bactericidal properties of titanium. Thus, with the use of quantitative photon counting microscopy, the inventors of the invention disclosed in the Swedish patent application no 0700457-5 were able to measure the chemiluminescent signal from MCLA (2-methyl-6-[p-methoxyphenyl]-3,7-dihydroimidazo[1,2-a]pyrazin-3-one) induced by production of superoxide from J774A.1 mouse macrophages stimulated with PMA (phorbol 12-myristate 13-acetate) and found a significant reduction by the crystalline phases.

In order to increase the efficiency of the porous titanium granules they may be transformed into the crystalline isoforms by heat treatment in inert atmosphere or vacuum. It is known that such transformation occurs at temperatures above 900° C., but well below the melting point of titanium (1668° C.) and in some instances as low as 500° C.

Therefore, according to one embodiment of the present invention, the particles, grains or granules are preheat treated in inert atmosphere or vacuum at a temperature of 500° C. or above, but below the melting point of the titanium, one or more titanium oxides or the titanium alloy or the mixture thereof.

As mentioned before, the value of the specific surface area of the particles, grains or granules according to the invention is in some cases of great importance, and could be regarded as a direct indication on the magnitude of the anti-inflammatory and/or antibacterial effects. However, as discussed above, there are other important factors as well for these effects. As disclosed in the examples below, there are different levels of specific surface area obtainable by different treatments of the particles, grains or granules according to the invention.

According to one embodiment of the present invention, the grains or granules are pre-treated with HF or H₂O₂ and have a mean specific surface area of at least 0.25 m²/g according to the BET method.

According to another specific embodiment of the present invention, the grains or granules are pre-treated with HF and have a mean specific surface area of at least 0.40 m²/g according to the BET method.

According to yet another specific embodiment of the present invention, said grains or granules are pre-oxidised and have a mean specific surface area of at least 0.40 m²/g according to the BET method.

Additional advantageous effects may be obtained by addition of substances that influence the biological environment. This could be achieved by filling or incorporating a porous body, i.e. a (particle), grain or granule, according to the invention with such substances or combinations thereof or by modification of the surface of the particle, grain or granule or the outer surface of the pores of such particles, grains or granules. This could for example be achieved by binding or attaching at least one substance to the surface of the particles or grains or to the surface of or into the pores of the particles or grains. By other modifications, including binding of biologically active substances, the implant may enhance tissue healing, remodelling or ingrowth.

Therefore, according to one embodiment of the present invention, at least one substance, which substance is biologically active, is filled into the pores of the particles, grains or granules and/or is bound to the surfaces of the particles, grains or granules.

These additional substances may e.g. be factors promoting tissue growth or regeneration, or be antibiotics. Therefore, according to one embodiment of the present invention, the at least one substance comprises or is chosen from the group of antibiotics, factors promoting tissue growth or regeneration or a combination thereof. Examples of such substances or factors are bone morphogenic factor, bisphosphonate, alfa-keto glutarate, simvastatin, Emdogain® (see below), gentamicin and synthetic type I collagen (as PepGen P-15). Another example is peroxides, e.g. a hydrogen peroxide solution, which already has been mentioned.

According to one specific embodiment of the present invention, the at least one substance, which is filled into the pores of the particles, grains or granules and/or is bound to the surface of particles, grains or granules, is at least one active enamel substance. An example thereof is Emdogain®, which is mentioned above and is a commercial product comprising amelogenins. Emdogain® is marketed by Biora AB and it comprises about 30 mg/ml of active enamel substance in propylene glycol alginate (PGA). This is a preferred amount in a possible pharmaceutical composition for incorporating, filling, attaching or binding to the implant grains according to the invention.

There are different ways of binding substances to the particles or grains, or modify the particles or grains, according to the invention. The physical and chemical surface modification methods can be categorised into three different types, the noncovalent coatings, the covalently attached coatings and modifications of the original surface.

The methods used for noncovalent coatings are preferably solvent coatings, surface-active additives or vapor deposition of carbons and metals, in which the latter one some covalent reaction may occur.

The preferred methods for covalently attached coatings are RFGD plasma deposition, in this case at low-pressure ionized gas environments typically at about ambient temperature, other plasma gas processes, gas-phase deposition, as chemical vapour deposition (CVD), chemical grafting and biological modification (biomolecule immobilization).

The methods for modifications of the original surface are preferably ion exchange, by chemical reactions, like non-specific oxidation, and conversion coatings.

Addition of factors that increase the initial adherence of the particles and/or granules to each other and to the surrounding tissue may also be added. These should preferably be resorbable, an example of this is fibrin. Other possibilities are to use synthetic adhesives like cyanoacrylate.

It should also be noted that a minor fraction of an implant according to specific embodiments of the invention could consist of titanium in combination with other materials than titanium, one or more titanium oxides or titanium alloy, e.g. allogenic bone, ceramics, polymers, adhesives.

Moreover, it should further be noted that there are several conditions comprising at least one weakened vertebra or one compression fracture of a vertebra, e.g. osteoporosis.

One object of the present invention is also to provide the use of particles, grains or granules or a mixture thereof, or an implant suspension according to the present invention. Therefore, according to one embodiment of the present invention there is provided use of particles, grains or granules or a mixture thereof, or an implant suspension comprising at least one resorbable fluid vehicle and particles, grains or granules or a mixture thereof, wherein the particles, grains or granules individually have a mean length from one side to the opposite side, through a geometrical centre, of up to 5 mm, for the manufacture of a medicament for the vertebroplastic treatment or kyphoplastic treatment of a condition comprising at least one weakened vertebra or one compression fracture of a vertebra.

Specific other embodiments of the present invention, in relation to the use of particles, grains or granules or a mixture thereof, or an implant suspension according to the present invention, for the manufacture of a medicament for the vertebroplastic treatment or kyphoplastic treatment of a condition comprising at least one weakened vertebra or one compression fracture of a vertebra, are disclosed above in relation to the specific embodiments of the method for treatment of such a condition according to the present invention.

Moreover, another object of the present invention is to provide particles, grains or granules or a mixture thereof, or an implant suspension comprising at least one resorbable fluid vehicle and particles, grains or granules or a mixture thereof, for the treatment of a condition comprising at least one weakened vertebra or one compression fracture of a vertebra. Therefore, according to one embodiment of the present invention there is provided particles, grains or granules or a mixture thereof, or an implant suspension comprising at least one resorbable fluid vehicle and particles, grains or granules or a mixture thereof, wherein the particles, grains or granules individually have a mean length from one side to the opposite side, through a geometrical centre, of up to 5 mm, for the vertebroplastic treatment or kyphoplastic treatment of a condition comprising at least one weakened vertebra or one compression fracture of a vertebra.

According to one specific embodiment of the present invention, the at least one resorbable fluid vehicle is chosen from the group consisting of NaCl (aq), hyaluronic acid, PEG, titanium peroxy gel, methyl cellulose, carbomethyl cellulose, dextran, a high viscous polymeric gel and a protein solution, or a combination thereof.

Other specific embodiments of the present invention, in relation to the particles, grains or granules or a mixture thereof, or the implant suspension according to the present invention, for the vertebroplastic treatment or kyphoplastic treatment of a condition comprising at least one weakened vertebra or one compression fracture of a vertebra, are disclosed above in relation to the specific embodiments of the method for treatment of such a condition according to the present invention.

### CONCLUSIONS

Due to the fact that there exists a great risk with the existing techniques of treating a condition comprising at least one weakened vertebra or one compression fracture of a vertebra, which is possible to occur with e.g. osteoporosis, there is a need for a new and more safe treatment. These risks with existing techniques, such as the today used vertebroplasty and kyphoplasty, involve the leakage of bone cement from a treated vertebra and into e.g. segmental veins and to the lungs and into the spinal canal. There is also a possibility to use other fluid phase compounds, and not only bone cement, but there exists a risk of leakage of these fluid phase compounds in this cases as well. The use of particles, grains or granules or a mixture thereof, or an implant suspension according to the present invention, instead of bone cement or these mentioned other fluid phase compounds, for the treatment of such a condition reduces or eliminates such risks.

1. Method for treatment of a condition comprising at least one weakened vertebra or one compression fracture of a vertebra, the method comprising:
injecting the vertebra by vertebroplasty with particles, grains or granules or a mixture thereof, or an implant suspension comprising at least one resorbable fluid vehicle and particles, grains or granules or a mixture thereof, wherein the particles, grains or granules individually have a mean length from one side to the opposite side, through a geometrical centre, of up to 5 mm.

2. Method for treatment of a condition comprising at least one weakened vertebra or one compression fracture of a vertebra, the method comprising:
performing kyphoplasty with a surgical balloon on the vertebra; and
injecting a cavity formed inside of the vertebra, after the balloon has been surgically removed, with particles, grains or granules or a mixture thereof, or an implant suspension comprising at least one resorbable fluid vehicle and particles, grains or granules or a mixture thereof, wherein the particles, grains or granules individually have a mean length from one side to the opposite side, through a geometrical centre, of up to 5 mm.

3. Method for treatment according to claim 1, wherein the particles, grains or granules are of any type of material that is a polymer, ceram, metal-ceramic mixture (composite), metal, metal alloy, metal oxide or a mixture thereof.

4. Method for treatment according to claim 1, wherein the particles, grains or granules comprise titanium, one or more titanium oxides or titanium alloy or a mixture thereof.

5. Method for treatment according to claim 1, wherein the particles, grains or granules are spherical and optionally individually have a bone inducting surface.

6. Method for treatment according to claim 3, wherein the particles, grains or granules are optionally spherical and are additionally provided with nanotubes of titanium oxide on their surfaces.

7. Method for treatment according to claim 1, further comprising:
performing a final additional sealing injection subsequent to the injecting, in which at least one mesh and/or irregular particles, grains or granules, optionally of titanium, one or more titanium oxides or titanium alloy or a mixture thereof, are injected into the vertebra or the cavity formed inside of the vertebra to form a sealing.

8. Method for treatment according to claim 1, wherein the at least one resorbable fluid vehicle is chosen from the group consisting of NaCl (aq), hyaluronic acid, PEG, propylene glycole alginate (PGA), titanium peroxy gel, methyl cellulose, carbomethyl cellulose, dextran, a high viscous polymeric gel and a protein solution, or a combination thereof.

9. Method for treatment according to claim 1, wherein the implant suspension is a gel having a melting temperature above ambient temperature and below 37° C. (body temperature), which gel optionally comprises at least one of NaCl (aq), hyaluronic acid, PEG, propylene glycole alginate (PGA), titanium peroxy gel, methyl cellulose, carbomethyl cellulose, dextran, a high viscous polymeric gel, or a protein solution.

10. Method for treatment according to claim 1, wherein the particles, grains or granules are irregular and optionally porous.

11. Method for treatment according to claim 1, wherein the grains or granules have a mean specific surface area of at least 0.15 m²/g according to the BET method.

12. Method for treatment according to claim 1, wherein the particles:
have a mean length from one side to the opposite side, through a geometrical centre, of <200 μm; and
wherein the grains or granules:
have a mean length from one side to the opposite side, through a geometrical centre, of at least 200 μm and up to 2 mm.

13. Method for treatment according to claim 1, wherein the particles, grains or granules are pretreated with at least one fluoric compound, hydrochloric acid, sulphuric acid, phosphorous acid, a peroxide compound chosen from the group consisting of hydrogen peroxide (H₂O₂) and organic peroxides, or oxalic acid, or a combination thereof, or dry etched with fluorinated or chlorinated gases.

14. Method for treatment according to claim 13, wherein the fluoric compound is any type of fluoric acid, hydrofluoric acid in combination with nitric acid, ammonium fluoride, ammonium bifluoride (also in combination with nitric acid), or hydrogen fluoride (HF).

15. Method for treatment according to claim 13, wherein the concentration is from 0.05 to 1.0% for fluoride acids, from 0.5 to 30.0% for hydrogen peroxides and from 0.2 to 20.0% for oxalic acids.

16. Method for treatment according to claim 13, wherein the concentration is about 0.2% for fluoride acids, about 30% for hydrogen peroxides and about 10% for oxalic acids.

17. Method for treatment according to claim 1, wherein the particles, grains or granules are pretreated by oxidation by heat treatment in oxidising atmosphere at temperatures between 20 and 1000° C. and/or by electrochemical procedure.

18. Method for treatment according to claim 1, wherein the particles, grains or granules are pretreated by anodical oxidation using spark erosion to increase the surface area.

19. Method for treatment according to claim 1, wherein the particles, grains or granules have been produced according to a spark erosion procedure where the particles, grains or granules have been brought in contact with an anode using a flexible net or porous sponge-like structure.

20. Method for treatment according to claim 1, wherein the particles, grains or granules are provided with nanotubes of titanium oxide, by a pre-treatment involving anodic oxidation, on their surfaces.

21. Method for treatment according to anyone claim 1, wherein the particles, grains or granules are provided with nanotubes of titanium oxide, by a pre-treatment involving anodic oxidation, on their surfaces, in an electrolyte solution comprising hydrofluoric acid (HF), potassium fluoride (KF), sodium fluoride (NaF), glycerol, (NH₄)₂SO₄ or (NH₄)F or a combination thereof.

22. Method for treatment according to claim 4, wherein
the particles, grains or granules are preheat treated in inert atmosphere or vacuum at a temperature of 500° C. or above, but below the melting point of the titanium, one or more titanium oxides or the titanium alloy or the mixture thereof.

23. Method for treatment according to claim 1, wherein the grains or granules are pre-treated with HF or H₂O₂ and have a mean specific surface area of at least 0.25 m²/g according to the BET method.

24. Method for treatment according to claim 1, wherein the grains or granules are pre-treated with HF and have a mean specific surface area of at least 0.40 m²/g according to the BET method.

25. Method for treatment according to claim 1, wherein the grains or granules are pre-oxidised and have a mean specific surface area of at least 0.40 m²/g according to the BET method.

26. Method for treatment according to claim 1, wherein at least one substance, which substance is biologically active, is at least one of filled into the pores of the particles, grains or granules and bound to the surfaces of the particles, grains or granules.

27. Method for treatment according to claim 26, wherein the at least one substance comprises antibiotics, factors promoting tissue growth or regeneration, or a combination thereof.

28. Method for treatment according to claim 27, wherein the at least one substance is bone morphogenic factor, bisphosphonate, alfa-keto glutarate, simvastatin, gentamicin or synthetic type I collagen, or at least one active enamel substance, or a combination thereof.

29. Method for treatment according to claim 1, wherein the condition comprising at least one weakened vertebra or one compression fracture of a vertebra is osteoporosis.

30. A method, comprising:
using particles, grains or granules or a mixture thereof, or an implant suspension comprising at least one resorbable fluid vehicle and particles, grains or granules or a mixture thereof as defined in claim 1, for the manufacture of a medicament for the vertebroplastic treatment or kyphoplastic treatment of a condition comprising at least one weakened vertebra or one compression fracture of a vertebra.

31. Method according to claim 30, wherein the condition comprising at least one weakened vertebra or one compression fracture of a vertebra is osteoporosis.

32. Particles, grains or granules or a mixture thereof, or an implant suspension comprising at least one resorbable fluid vehicle and particles, grains or granules or a mixture thereof, wherein the particles, grains or granules individually have a mean length from one side to the opposite side, through a geometrical centre, of up to 5 mm,
for the vertebroplastic treatment or kyphoplastic treatment of a condition comprising at least one weakened vertebra or one compression fracture of a vertebra.

33. Particles, grains or granules or a mixture thereof, or an implant suspension comprising at least one resorbable fluid vehicle and particles, grains or granules or a mixture thereof according to claim 32, wherein the condition comprising at least one weakened vertebra or one compression fracture of a vertebra is osteoporosis.

34. Method for treatment according to claim 2, wherein the particles, grains or granules are of any type of material that is a polymer, ceram, metal-ceramic mixture (composite), metal, metal alloy, metal oxide or a mixture thereof.

35. Method for treatment according to claim 2, further comprising:
performing a final additional sealing injection subsequent to the injecting, in which at least one mesh and/or irregular particles, grains or granules, optionally of titanium, one or more titanium oxides or titanium alloy or a mixture thereof, are injected into the vertebra or the cavity formed inside of the vertebra to form a sealing.
